(19) United States
(12) Patent Application Publication
(10) Pub. No.: US 2007/0154465 A1
(43) Pub. Date: Jul. 5, 2007

(54) Stem cell therapy for retinal disease

(76) Inventors: Alexandar Kharazi, San Diego, CA (US); Nikolai Tankovich, San Diego, CA (US); Basil Hantash, E. Palo Alto, CA (US)

Correspondence Address: STEMEDICA CELL TECHNOLOGIES, INC, 5375 MIRA SORRENTO PLACE, SUITE 100, SAN DIEGO, CA, 92121, US

(21) Appl. No.: 11/323,714

(22) Filed: Dec. 30, 2005

(51) Int. Cl. A61K 48/00; C12N 5/08; A61N 5/00

(52) U.S. Cl. 424/93.21; 435/368; 607/87

(57) ABSTRACT

Method of treating retinal disease by intravitreally injecting defined cytokine-transfected stem cell population; adipose-derived stem cell comprising transient wound-healing trangenes; defined homogeneous and heterogeneous populations of cytokine-transfected adipose-derived stem cells; method of treating retinal disease involves step of laser-inducing retinal injury followed by intravitreally injecting a population of stem cells comprising transiently transfected stem cells into the eye of the mammal in a number sufficient for repopulation, intraretinal integration and differentiation into normal photoreceptor cells.

### BACKGROUND OF THE INVENTION

Retinitis pigmentosa is a collective term for a group of related, yet distinct, progressive dystrophies of the photoreceptors and the pigment epithelium that are a major cause of blindness. In the disease, there is concomitant attenuation of the retinal vasculature. It is thought that vascular loss follows decreased metabolic demand by the photoreceptors. Currently no definitive treatment for retinitis pigmentosa exists. Stem cell treatment approaches have been recently reviewed [2].

The use of stem cell technology appears to be especially attractive because it provides the ability not only to halt disease progression but also to reverse the loss of function through replacement of lost photoreceptors and accessory cells. However, even in the face of recent promising results, it is generally agreed that retinal item cell replacement methods still suffer from the problem of failed or incorrect functional integration of grafted cells [1].

In a number of studies involving neuronal stem cell transfer [3, 4, 5], researchers reported that only a fraction of injected stem cells expressed neuron-specific markers and it was hard to draw any conclusion on changes in the visual function of the recipients following transplantation. Stem cell transplantation treatment faces the problems of cell survival, proliferation, and phenotypic maturation of stem cells, particularly into functional neurons and especially photoreceptors.

### SUMMARY OF THE INVENTION

The present invention provides a method of treating retinal disease with stem cells. The method comprises the step of intravitreally injecting a stem cell population into the eye of a mammal in need of treatment. The stem cell population comprises stem cells which have been genetically modified ex vivo by transient transfection with wound-healing cytokine transgenes. The stem cell population is an adipose-derived stem cell population. The transient transgenes are selected from the group of pro-inflammatory and wound-healing cytokines, including, but not limited to IL-6, IL-12, LIF, IFN(s), EGF, VEGF, FGF-1, and FGF-2. One embodiment of the inventive cells involves co-transfection using transient transgenes EGF and FGF-2. Still other embodiments involve singly transfected stem cells.

A variation of the method involves, prior to intravitreally injecting the isolated stem cells into the eye of the mammal, the step of selectively injuring the site of retinal lesion with photodynamic therapy [laser] to promote engraftment of the donor inventive cells.

In another aspect, the invention is directed to an isolated adipose derived stem cell comprising transient wound-healing trangenes. The transgenes are selected from the group consisting of EGF, FGF-2, [see above]. A useful embodiment of the inventive cell incorporates the transient transgenes EGF and FGF-2.

Another aspect of the invention involves a substantially homogenous population of adipose-derived stem cells comprising a plurality of a stem cells which comprises transient wound-healing transgenes, the transgenes selected from the group consisting of EGF, FGF-2, one embodiment involving transgenes EGF and FGF-2.

### BRIEF DESCRIPTION OF DRAWINGS

FIG. 1 is a plot of the ratio of reflectivity.

FIGS. 2A and 2B illustrate the structure of laser levels of Nd³⁺ ion in YAG and YAP crystals.

FIG. 3 depicts a rotating mirror mount.

FIG. 4 illustrates a plot used in a technique for selecting single lines.

FIG. 5 is a plot of absorption coefficient vs. wavelength showing absorbtion of 1079 nm wavelength in melanin, oxyhemoglobin and water.

FIG. 6 is a diagram of a cooling device which provides prior simultaneous and subsequent cooling.

FIG. 7 is a scheme of a general protocol for preparation and use of the inventive cells and defined cell populations.

### DETAILED DESCRIPTION OF THE INVENTION

### DEFINITIONS

The term “stem cells” as used herein means: (Zuk et al. p. 4292) cells possessing self-replicating potential and the ability to give rise to terminally differentiated cells of multiple lineages (Hall and Watt, 1989). Stem cells are capable of generating identical progeny through unlimited numbers of cell divisions whilst retaining the ability to respond to physiological demands by producing daughters committed to differentiate.

The term “transgene” means a gene which has been transferred from one organism or cells thereof to another organism or cell thereof using recombinant DNA techniques.

The term “transient transgene” as used herein means a transgene that is transiently expressed, i.e. will not be replicated following the cell division.

The term “wound-healing gene” as used herein means a gene encoding a protein which is involved in the wound-healing process.

The term lipoaspirate refers to adult adipose tissue as a source of stem cells. Zuk et al. [6] has demonstrated stem cell populations within human lipoaspirates. This cell population, called processed lipoaspirate (PLA) cells, can be isolated from adipose tissue in significant numbers and exhibits stable growth and proliferation kinetics in culture, as well as multilineage differentiation capacity. The term “adult” in reference to adipose tissue, includes adipose tissue isolated postnatally, i.e., from juvenile and adult individuals, as opposed to embryos. The term “adult mammal” refers to both juvenile and fully mature mammals.

As used herein, the term “treating” means injecting the isolated stem cells into an eye of a mammal in a number sufficient to ameliorate the effects of the retinal disease. Depending on the context of the retinal disease, amelioration encompasses the terms “inhibiting retinal neuronal degeneration,” “rescuing neuronal networks in the retina,” “rescuing blood vessels in the retina,” “promoting retinal neovascularization,” “inducing photoreceptor rescue.”

As used herein, and in the appended claims, singular indicators (eg., “a” or “one”) include the plural, unless otherwise indicated.

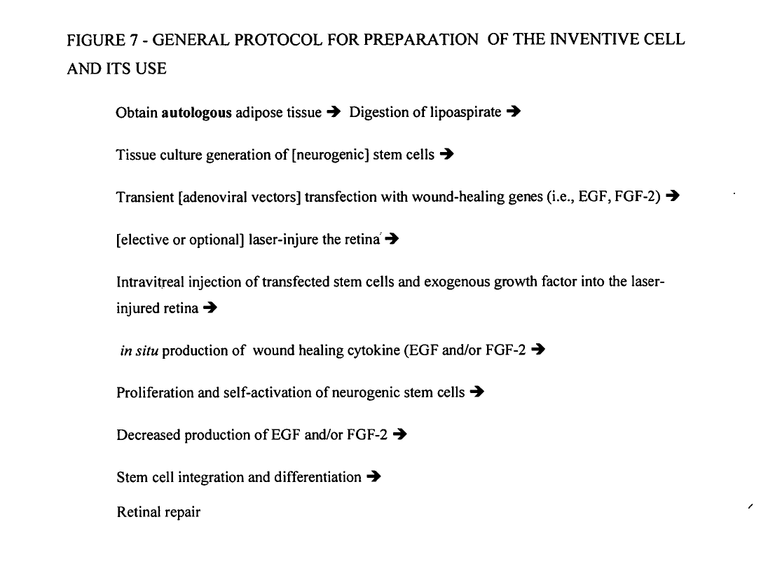

The method of the invention involves an adipose-derived stem cell which has been transiently transfected ex vivo with one or more genes which encode wound-healing cytokines. The general protocol for preparation and use of the inventive cell is shown in FIG. 7.

While the transfected cell of the invention can be solitary and isolated from other cells, preferably, for clinical use, it is within a population of cells consisting essentially of the inventive lipo-derived stem cells. In certain embodiments, the invention provides a defined population of the inventive cells. For example, derived from a clone of lipo-derived stem cell, a first stem cell is transfected with a first wound healing gene; a second stem cell is transfected with a second, different wound healing gene. The first and second transfected stem cells are separately expanded. A defined population of the first and/or second transfected stem cells is then made from the expanded populations. The defined population may be substantially homogeneous, comprising solely the first transfected stem cells or the second transfected stem cells. Other embodiments of the defined populations of the invention include mixtures of the first and second transfected stem cells. Embodiments of mixed defined populations include populations which comprise proportions in which the ratio of first to second transfected stem cells is between 1:99 and 99:1.

In other embodiments, the defined population of inventive cells is combined with peripheral blood-derived progenitor cells, for example, CD106 or CD 10+BMS.

In general, the inventive lipo-derived cells are genetically modified to transiently express exogenous wound-healing genes. Thus, the invention provides a method of genetically modifying such cells and populations. In accordance with this method, the cell is exposed to a gene transfer vector comprising a nucleic acid including a transgene, such that the nucleic acid is introduced into the cell under conditions appropriate for the transgene to be transiently expressed within the cell. The transiently-expressed transgene generally is an expression cassette, including a coding polynucleotide operably linked to a suitable promoter. Within the expression cassette, the coding polynucleotide is operably linked to a CMV (cytomegalovirus) promoter.

The coding polynucleotide encodes a cytokine protein. Thus, for example, the coding polynucleotide encodes a gene expressing “wound healing cytokine(s)”. Of course, where it is desired to employ gene transfer technology to deliver a given transgene, its sequence will be known.

The expression cassette containing the transgene is incorporated into an adenoviral vector. The adenoviral vector is introduced into the target stem cells by infection. The genetically altered cells can be employed as bioreactors for producing the product of the transgene. In other embodiments, the genetically modified cells in defined populations are employed to deliver the transgene and its product to an animal. For example, a defined population of the transfected cells, once genetically modified, can be introduced into the animal under conditions sufficient for the transgene to be expressed in vivo.

In certain embodiments, the transiently transfected cells and defined populations of the present invention express and secrete epidermal growth factor (EGF), fibroblast growth factor-2 (FGF-2), or both. EGF is a potent growth factor that stimulates the proliferation of various epidermal and epithelial cells. Additionally, EGF has been shown to be involved in wound healing. FGF-2 is a single-chain polypeptide growth factor that plays a significant role in the process of wound healing and is a potent inducer of angiogenesis. It is also an extremely potent inducer of DNA synthesis in a variety of cell types from mesoderm and neuroectoderm lineages. For the present invention, either or both EGF and FGF-2 are used as cytokines involved in proliferation and self-renewal of neuronal stem cells.

Accordingly, the transiently transfected cells and populations of the present invention are genetically modified to secrete EGF, FGF-2 or both cytokines in vitro in defined populations and immediately following intravitreal inoculation.

Obtaining Adipose Tissue—the Lipoaspirate

The stem cells of the invention can be obtained from adipose tissue by any suitable method. A first step in any such method requires the isolation of adipose tissue from the source animal or humans. Typically, human adipose stromal cells are obtained from living donors, using well-recognized protocols such as surgical or suction lipectomy. Indeed, as liposuction procedures are so common, liposuction effluent is a particularly preferred source from which the inventive cells can be derived.

Generation of Neurogenic Stem Cells

The neurogenic stem cells are generated from autologous adipose tissue using methods well known in the art for producing multipotent stem cells from lipoaspirate and inducing neurogenic differentiation in vitro [6] and see U.S. Pat. No. 6,777,231. In one protocol, a lipoaspirate specimen is enzymatically digested in a digestion solution (0.1% solution of Collagenase type I in Hank's balanced salt solution). 20 ml of this solution per estimated 1 cm3 of specimen is used for processing. The specimen is washed in phosphate buffered saline and transferred into the stirring flask with the digestion solution. The flask is kept in 37° C. water bath on the magnetic stand for 30 min with gentle stirring. The digestion product is filtered through a single layer of sterile Nitex screen and centriguged for 10 min at a low speed. The cell pellet is re-suspended in Neurobasal™ medium with B-27 Supplement (Invitrogen) containing recombinant Epidermal Growth Factor, EGF (20 ng/ml) and Fibroblast Growth Factor-2, FGF-2 (10 ng/ml). The cells are analyzed by flow cytometry for the expression of stem cell markers, including CD29, CD44, CD71, CD90, CD49d, CD13, CD73, CD105, CD166, HLA-ABC (while being negative for HLA-DR and CD49D for bone marrow although CD49d may be positive in fat derived stem cells), seeded in tissue culture plates at the density of 104 cells/ml and cultured in 5% CO2 incubator at 37° C. The cells are harvested when at least 80% of the population expresses immature neuronal markers (NSE, NeuN) but before the expression of mature neuronal markers such as MAP-2 or NF-70 as determined by flow cytometry.

Transient Transfection with Adenoviral Vectors

The next step involves making transplanted stem cells produce cytokines, which enhances their post-injection survival and provides the initial proliferation signal. In symmetrical division of neuronal stem cells, it is known that a combination of EGF and FGF-2 is sufficient to initiate and maintain continuous proliferation. Accordingly, in certain embodiments, the neuronal stem cell enriched population is transiently transfected with adenoviral vectors for transfecting the stem cells with an EGF gene, an FGF-2 gene, or both genes.

The transience is achieved by using adenoviral vectors, which do not have a replication origin, so the cytokine secretion will decrease with each division of a transfected cell. The specific details of a transfection procedure are described below.

Expansion and Plasticity

Techniques for isolating and expanding mesenchymally derived adult stem cells are well known in the art [6] and Handbook of Stem Cells, ed. Robert Lanza, 2004, Elsevier Inc.

Cell plasticity in the course of expansion can be maintained by exogenous of leukemia inhibitory factor (LIF) using techniques well known in the art Handbook of Stem Cells, ed. Robert Lanza, 2004, Elsevier Inc.

The timing of triggering differentiation in the course of expansion is achieved or directed by the exogenous addition of growth factors well known in the art (Handbook of Stem Cells, ed. Robert Lanza, 2004, Elsevier Inc.).

Vector Preparation and Purification

Plasmid clones for the human cytokines, epidermal growth factor (beta-urogastrone, EGF) and fibroblast growth factor 2 (bFGF) (available from Origene (Catalog Numbers TC1278404 and TC118884 respectively)) The plasmids are expanded in E. coli and extracted by using the S.N.A.P. Prep Kit (Invitrogen). The genes containing the sequences for the cytokines are extracted by PCR using primers for the corresponding cytokines. After isolation the genes are ligated into the entry vectors purchased from Invitrogen.

The newly constructed entry clones are expanded in E. coli using media containing the appropriate antibiotic. The entry clones are extracted and analyzed for purity. The genes inserted into the entry clones are excised and ligated to the destination vector by performing a Gateway LR recombination reaction which creates the adenoviral expression clone. The expression clone is transformed into E. coli. The expression clone is expanded, extracted and digested using restriction enzyme PAC I to expose the inverted terminal repeats (ITRs).

The digested expression clone is transfected into the 293A producer cell line (Invitrogen) for the initial expansion. The cells are lysed, and the lysate is used to infect the 293A producer cells for the secondary expansion. The media is replaced every 2-3 days for approximately 7-10 days until visible regions of cytopathic effect (CPE) (ie. Plaques) are observed. Infections will be allowed to progress until 80% of CPE is observed (about 10-13 days). The cells are harvested and placed in microcentrifuge tubes. The tubes are incubated at −80° C. for 30 minutes after which the tubes are incubated at 37° C. for 15 minutes. Again the tubes will be incubated at −80° C. for 30 minutes is collected and aliquoted to create a Master Virus Bank. The vials are frozen at −80° C. until further use.

Vector Lot-Release Preparation

A vial from the Master Virus Bank is removed, thawed at 37° C. and 100 μl of virus is placed in a flask containing the 293A producer cell line at a 80-90% confluency. The cells are incubated at 37° C. for 2-3 days post infection. The cells are harvested and placed in micro centrifuge tubes. The tubes are incubated at −80° C. for 30 minutes after which the tubes are incubated at 37° C. for 15 minutes. Again the tubes are incubated at −80° C. for 30 minutes followed by an incubation of 15 minutes at 37° C. The tubes are centrifuged, and the supernatant is collected and aliquoted to create a lot-release. Although separate lots are prepared for each cytokine, the same lot can be used for different patients. The vials are frozen at −80° C. until further use.

Titration of Adenoviral vector

After a lot-release vial is thawed, a 10-fold serial dilution is prepared starting from 10-4 to 10-9. For each dilution the construct is dissolved in complete media to a final volume of 1 ml. The dilutions are added to the previously plated 293A cells. The cells are incubated overnight. The following day a 4% agarose over lay solution is applied to the 293A cells. The cells are incubated at room temperature for 15 minutes. The cells are returned to the 37° C. incubator. On day five following initial plate seeding, 1 ml of additional 4% overlay solution is added to the cells followed by a 15 minute incubation at room temperature. The cells are returned to the 37° C. incubator and observed for 8-10 days post-infection. On approximately 10-14 days following infection, a dye solution is applied to the cells and incubated at 37° C. for 3 hr. The plates are then removed from the incubator and the visible plaques are counted to determine the titer for the specified lot of virus. The dilution that gives a number in the range of 1×10⁸ to 1×10⁹ pfu/ml is suitable for use in further applications.

Transfection of Neuronal Stem Cells

A six-well plate is seeded with the stem cells harvested as described above [see section Generation of Neurogenic Stem Cells] and incubated for 24 hr at 37° C. On the day of transfection a vial from the specified lot-release to be used is thawed. The contents of the vial are diluted for delivering a suitable titer. The virus solution is added to the target stem cells and incubated overnight at 37° C. The following day the media is removed and replaced with fresh complete culture media. The cells are harvested 48 hr post-transduction and frozen down. The culture supernatant are collected for protein expression assay (see below).

Validation of Transfected Stem Cell Lot-Release

1. Sterility Assays.

The aliquots of each lot of transfected stem cells are submitted for general sterility testing, Mycoplasma testing and endotoxin testing. Only lots that pass those tests are used.

2. Cytokine Production Assays.

Assays are performed for production by transfected stem cells of cytokines EGF, FGF-2, or both. The concentration of cytokines in the supernatants of transfected stem cells are determined by ELISA, an in vitro enzyme-linked immunosorbent assay for the quantitative measurement of human EGF/FGF-2 in serum, plasma, cell culture supernatants and urine. This assay employs an antibody specific for human EGF/FGF-2 coated on a 96-well plate. Standards and samples are pipetted into the wells and EGF/FGF-2 present in a sample is bound to the wells by the immobilized antibody. The wells are washed, and biotinylated anti-human EGF/FGF-2 antibody is added. After washing away unbound biotinylated antibody, HRP-conjugated streptavidin is pipetted to the wells. The wells are again washed, a TMB substrate solution is added to the wells and color develops in proportion to the amount of EGF/FGF-2 bound.

Intraorbital Injection.

An aliquot of a defined population containing about 5×10⁵ to 1×10⁶ validated neuronal stem cells which have been transiently transfected with EGF, FGF-2, or both and which are transgenically expressing EGF, FGF-2 or both are injected intravitreally into the laser-treated retina using syringe with a 33-G needle. The injection is performed 5 days following laser treatment. The number of stem cells injected into the eye is sufficient for arresting the disease state of the eye. For example, the number of cells can be effective for repairing retinal damage of the eye, stabilizing retinal neovasculature, maturing retinal neovasculature, and preventing or repairing vascular leakage and vascular hemorrhage.

In embodiments which employ the growth factors EGF and FGF-2, the protocol involves expanding a substantially homogeneous population of stem cells from a stem cell transiently transfected with either the EGF gene or the FGF-2 gene. In the case of intraorbitally injecting a defined population of cells transfected only with the EGF gene, FGF-2 is exogenously supplied (e.g. by intraorbital injection) to the transplanted cells at about the same time or soon after transplantation. Similarly, in the case of intraorbitally injecting a defined population of cells transfected only with the FGF-2 gene, EGF is exogenously supplied (e.g. by intraorbital injection) to the transplanted cells at about the same time or soon after transplantation.

By reference to the stem cell literature, one of skill in the art can determine which pairs of cytokines (e.g. EGF and FGF-2) are optimal for differentiating stem cells to histotypic states suitable for engrafting in a host site. Using the methods of the present invention, a substantially homogeneous population of stem cells from a stem cell transiently transfected with one of a pair of cytokines and the exogeneous provision of the other cytokine of the pair are provided, as above, to a patient suffering from a retinal disease.

Laser Retina Preparation to Promote Engraftment of the Donor Inventive Cells.

The use of the laser is to prepare a stem cells engraftment in to the retina by micro-injuries to release inflammatory and wound-healing cytokines from the retina tissue cells.

Laser injury includes a dual wavelength partially distributed light irradiation of retina. The first wavelength has a minimal absorption in pigmented cells and water and can penetrate deep into the retina's tissue up to 2-3 mm. The second wavelength has a specific absorption by retina's blood hemoglobin and can micro-coagulate small vessels. These two micro-injuries release inflammatory and wound-healing factors from different zones of retina and would attract stem cells to restore retina's cells as well as its blood vessels.

The first wavelength should have a length of from 810 nm to 1200 nm. Spot size less than 1 mm. Energy per pulse between 50 mW to 3 W with a pulse duration from 100 us to 500 ms. Spot density varies from 1 to 1000 per square centimeter.

The second wavelength has to be between 500 nm to 599 nm. Spot size less than 2 mm. Energy per pulse between 250 mW to 3 W with a pulse duration from 150 us to 500 ms. Spot density varies from 1 to 150 per square centimeter.

In the first embodiment the first and the second wavelengths are delivered simultaneously using Perovskite diode pumped solid state laser. The first wavelength is 1080 nm that has a minimal absorption in water and melanin and a doubled frequency wavelength is generated at 540 nm. Perovskite is the only laser that can generate two near infrared wavelength without restriction. It allows generate two wavelengths at 1080 and 1341 nm, make double frequency modulation, then mixed it and to get 590 nm wavelength with max absorption hemoglobin. The similar effect but by switching a laser from one wavelegth to the mode with another wavelength can be achieved by using Nd:YAG laser rod that generated 1064 nm and 532 nm respectively.

The present invention provides a laser system in which the gain medium is an excited YAP:Nd crystal. The system is configured so that the crystal produces a twin laser beam comprising wavelengths at both 1079 nm and 1341 nm with substantial intensities at each wavelength. Optical components are described which establish the desired ratio of the intensities of the light at each of the two wavelengths. These ratios, I_{1079 nm}/I^{1340 nm}, may vary from about 0.1 to 10. In a preferred embodiment of the invention a kit including a YAP:Nd crystal and a specially coated output coupler is provided for converting an existing Nd:YAG laser system to a twin light laser capable of producing the above described twin laser beam. The Nd:YAG laser system is unable to produce simultaneously 1064 nm and 1320 nm at substantial intensities of both wavelengths. In another embodiment a special combination output coupler is provided which contains at least three partially reflecting mirror elements, one coated to reflect a substantial fraction of light at 1079 nm and pass light at 1340 nm, another mirror coated to reflect a substantial fraction of light at 1340 nm and pass light at 1079 nm and a third mirror coated to reflect a substantial fraction of light at both wavelengths. In one embodiment the mirror elements are mounted on a rotating frame so that the desired mirror element can be in the beam path to define the resonant cavity. By switching between mirrors the laser operator is able to produce laser beams at 1079 nm, 1340 nm or to produce a beam at a both wavelengths. A preferred embodiment produces a pulsed laser beam capable of providing fluences on the retinal surface in the range of about 10 J/cm² to 200 J/cm² during a treatment period of less than 4 seconds.

A preferred application of this laser system is ophtalmology in which the two-wavelength beam illuminates the retina and heats the retinal relatively uniformly to a depth of a few millimeters. The eye surface can be cooled during the process to prevent or minimize surface tissue damage while tissue beneath the surface is altered due to thermal effects.

This application also discloses techniques for producing other wavelengths from the two-wavelength light produced by the YAP:Nd crystal.

Simultaneous Lasing

A pulsed laser beam is produce with a YAP:Nd crystal rod 2. Crystal rod 2 is pumped with a pump source (in this case a flash lamp, not shown) driven by a power supply, also not shown. An output coupler 4 is specially coated to partially reflect at both 1341 nm and 1079 nm to produce a laser beam with both wavelengths. The output coupler 4 and a maximum reflectance mirror 6 define the laser resonant cavity. Pulse durations are from about 10 to 20 milliseconds. The configurations should preferably be designed for operator selected pulse rates between 0.5 Hz and 100 Hz. In typical operation the laser is operated in bursts of pulses with each burst containing several pulses (such as 3 to 15 pulses) at selected pulse repetition rates. Preferably the controls are configured so that the operator can select a burst repetition rate up to about 2 Hz. Thus the operator could select a pulse repetition rate of 100 Hz with 5 pulses per burst and a burst repetition rate of 2 Hz. This would provide 10 pulses per second.

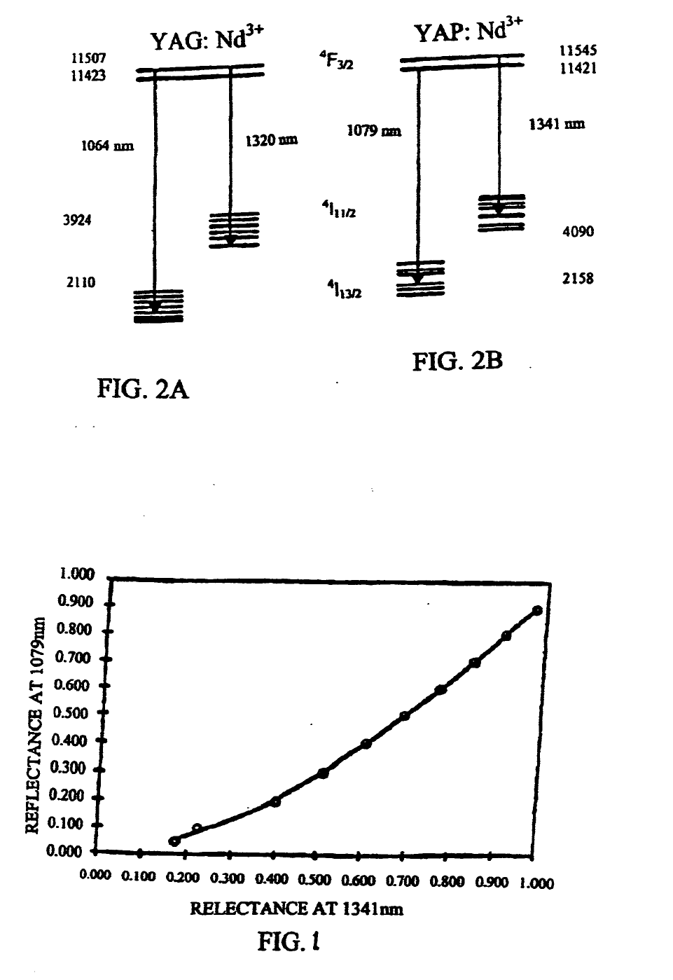

The high reflectivity mirror HR should have reflectance more 99.5% at both wavelength 1079 nm and 1341 nm. The output coupler mirror has a special coating enabling simultaneous lasing at 1079 and 1341 nm. It lies in the range 90-5% for 1079 and 97-17% for 1341 nm. The ratio of reflectivity may be chosen based on the plot shown in FIG. 1 calculated by the following formula:
ln(1/R₁)=2 L((σ₁ν₂/σ₂ν₁)α₂−α₁)+σ₁ν₂/σ₂ν₁ ln(1/R₂)
where:

R1 and R2 are the reflectivity of the mirror at 1079 and 1341 nm, and

σᵢ, νᵢ and αᵢ are stimulated emission cross section, frequency of the transition and passive loss in crystal all corresponding to two wavelengths.

Stimulated emission cross sections in the YAP crystal is 4.6×10⁻¹⁹ cm⁻² for the ⁴F_{3/2}-⁴I_{11/2} 1079 nm transition and 2.2×10⁻¹⁹ cm⁻² for the ⁴F_{3/2} -⁴I_{13/2} 1341 nm transition. The single pass linear loss depends on the quality of crystal. In this example they are taken to be 0.004 cm⁻¹ and 0.005 cm⁻¹ at 1079 and 1341 nm, respectively. FIGS. 2A and 2B illustrate the structure of laser levels of Nd³⁺ ion in YAG and YAP crystals. In the Nd:YAP crystal the upper laser level for 1079 nm and 1341 nm lines are significantly separated. This in part accounts for less competition between the two laser lines of the Nd:YAP crystal in comparison with Nd:YAG crystal. As a result simultaneous lasing at two wavelengths in YAP crystal is more efficient and easier to achieve. For approximately equal output intensities at each of the two wavelengths, reflectivities of the output couple mirrors should be about 40% for 1079 nm and about 80% for 1341 nm. Reflectivity of the high reflector mirror should be high at both wavelengths. In order to decrease losses of the laser light in the delivery system all transmission elements should preferably have special coatings to minimize reflectivity at 1079 nm and 1341 nm.

### Changing the Ratio Without Changing the OC

In the output of such a laser it is possible to change the ratio between 1079 and 1341 nm without changing the output coupler. One way is to introduce a dichroic linear absorbing filter or a polarizing filter in the beam train. The dichroic or polarizing filter is preferably placed in the handpiece. This enables the operator to switch wavelength just by changing a filter in the handpiece to a filter most suited for specific application. Or separate handpieces, each with different filters could be provided.

### Surface Cooling

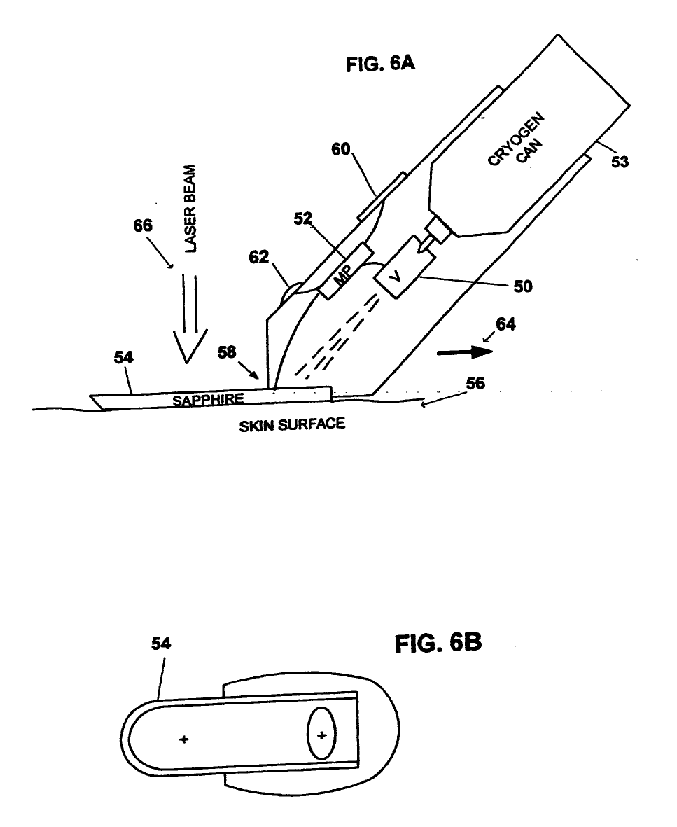

Fluencies in excess of 50 J per cm² when applied in a short period can cause severe damage to the retinal surface. However, damage can be avoided or minimized with prior, simultaneous or immediately subsequent cooling of the corneal surface. In this preferred embodiment corneal surface cooling is provided by cooling device 80 shown in FIG. 6 which provides prior simultaneous and subsequent cooling. One such device is described in U.S. Pat. No. 6,059,820 which is incorporated herein by reference. A short description is provided below.

A sapphire cooling window 54 is cooled by a spray from a liquid nitrogen can 53 through valve 50 controlled by microprocessor 52. A thermocouple 58 provides a temperature signal that is converted into a temperature value by microprocessor 52 for display on monitor 60. An off-on button is 62. In a preferred procedure the operator slides the cooling device in direction 64 along the corneal surface with one hand and applies laser pulses with applicator 70 using the other hand. Surface cooling can also be provided with an evaporating spray such as liquid nitrogen, air or tetrafluorethane.

Cooling device 80 protects the surface from damage and portions of the cornea below about 1 mm are not damaged because the penetration below 1 mm is not substantial. With this technique tissue at depths in the range of about 1 mm are damaged.

Separately Obtained Wavelengths from One Crystal in One Laser Box

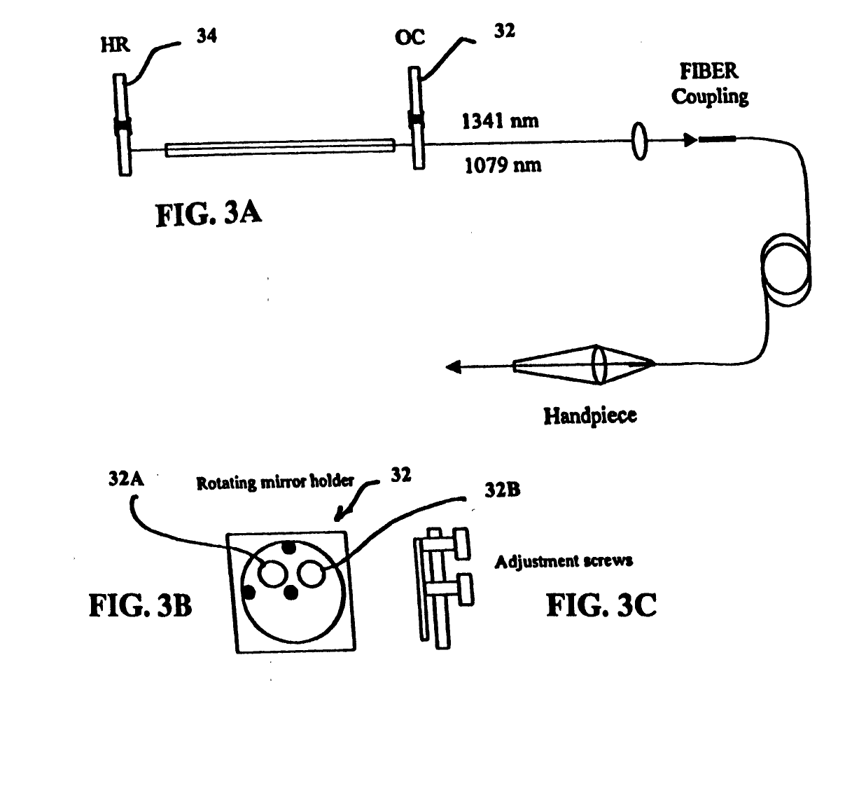

If simultaneous lasing at 1079 nm and 1341 nm is not desired the two wavelengths could be obtained separately from one crystal in one laser box. To do this the output coupler is provided with two additional mirrors. One is specifically made to reflect very preferentially at 1079 nm. The other is specifically made to reflect very preferentially at 1341 nm. A special output mirror holder is provided so that these mirrors can be interchanged, for example by linear translating or rotating. In order to make this approach workable special requirement for mirror mount should be met. The angular misalignment of laser resonator should be not worse than 10 arc second after changing mirrors. These kind of mirror holder are available from Newport Corporation with offices in Irvine Calif. A rotating mirror mount is depicted in FIG. 3.

### Dispersion Selection of Single Lines

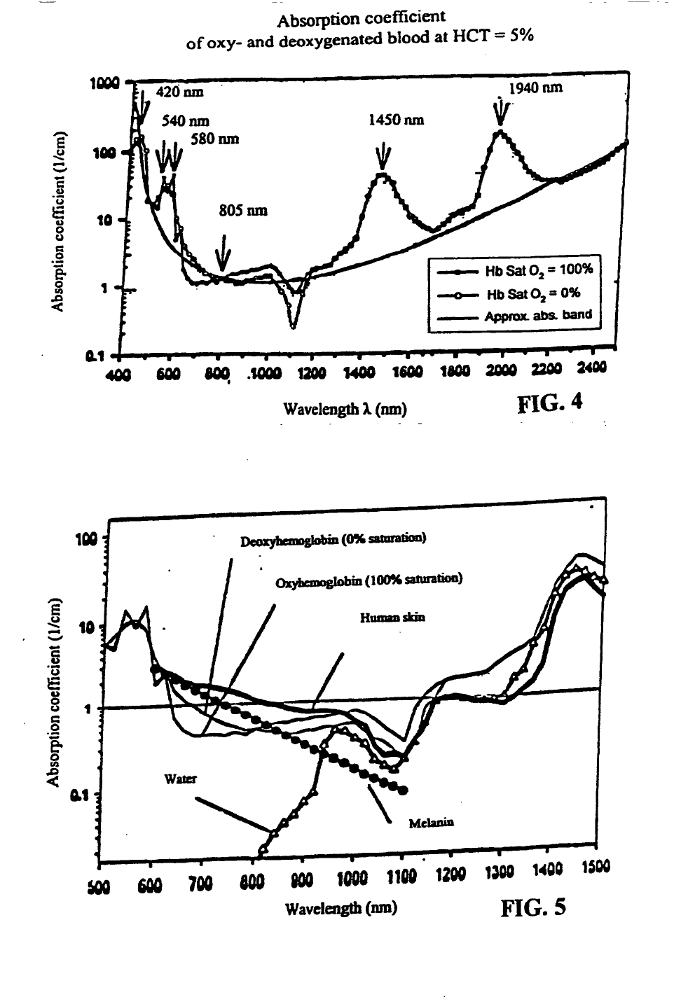

Single lines can also be selected using the technique shown in FIG. 4. A prism is placed between crystal 2 and maximum reflection mirror 6. The prism disperses the beam spectrally so that either of the lines can be selected by proper rotation of mirror 6. The prism should be made of a material with high optical dispersion in visible, for example flint-glass.

As it was noted above simultaneous operation on two wavelengths is much easier to achieve in Nd:YAP laser in comparison with Nd:YAG. Based on the approach described above it is possible to enhance laser performance of existing cosmetic Nd:YAG lasers in by substituting Nd:YAG crystal with a Nd:YAP crystal of the same dimensions and changing some delivery optics to enable transmittance of both 1079 and 1341 nm laser light. The procedure might be done in the field right in the doctor's office

### Second Harmonic

Each of the wavelengths available from the YAP:Nd crystal can be frequency doubled to provide additional wavelengths. Usually there is no need to filter the fundamental wavelength. Both fundamental and second harmonic wavelengths may be used for treatment simultaneously.

### Optical Components

The various optical components needed to fabricate the laser system described above are available from normal optics suppliers and techniques for arranging the components are well known to persons skilled in the laser-optics art. For example the YAP:Nd and YAP:Er rods for production of the 1079 and 1341 nm beams are available from Crytur, Ltd. with offices in Palackeho175, 51101 Turnov, Czeck Republic and Scientific Material Corp. with offices in Bozeman, Mont. To obtain output energy described above the preferred dimensions of YAP laser rods are 5×127 mm. Pump chamber for YAP:Nd lasers rods are available from Kigre Inc., Hilton Head Island, S.C. or LMI Corporation, Las Vegas, Nev. Optics for arranging the resonator cavities are available from CVI Corp. with offices in Albuquerque, N. Mex. Flash lamp pumps for these crystal rods are Xe flash lamps, for example model L8524 available from Perkin Elmer with offices in Sunnyvale, Calif. A power supply to drive flash lamps is available from Nada Electronics, UK or ASTEX Inc. with offices in Woburn, Mass. Mirrors optics and optics are available from CVI Corp.

### Preferred Specifications

The power supply and the flash lamp pump source and crystal rod should be sized for pulse energies of 22 J per. Energies per pulse at the other wavelengths are preferably about 4 J. The beam diameters prior to coupling into the optical fiber optic should be about 2 mm or less. The beams are normally focused onto the retinal surface to produce fluences in the range of about 30 to 90 J per cm² during short treatment period. Fluencies in excess of 50 J per cm2 could cause severe corneal damage. However, as indicated above, damage can be avoided or minimized with prior, simultaneous or immediately subsequent cooling.

### Treatment Wavelengths

With this one laser system a large variety of laser treatments can be provided. The wavelength 1079 nm is slightly absorbed in melanin, oxyhemoglobin and water. Thus, this beam is preferred for coagulation of deeper layers of retina tissues to create wells as a stromal scaffold formation. The 540 nm wavelength is more highly absorbed in hemoglobin than the 1079 nm wavelength so the 540 nm beam is good for superfacial retina's blood vessels coagulation and light treatment. Beams with the combination of 1079 nm and 540 nm beams wavelengths work well for retina preparation before stem cells administration and treatment. Cornea surface cooling before, during and after is preferred.

Results:

The method of treating retinal disease disclosed herein demonstrates that intravitreally injected compositions of a substantially homogenous population of adipose-derived stem cells [which comprise one or both of transient transgenes EGF and FGF-2] migrate into the retina, participate in the formation of normal retinal photoreceptor cells, and stabilize endogenous degenerating neuronal retina, pigment epithelium, and vasculature in a subject suffering from retinitis pigmentosa.

It should be understood that the invention involves standard techniques well known in the art for isolating, propagating, genetically modifying, and transplanting the inventive cells as a regenerative strategy with application to retinal disease. The inventive method achieves inhibition of cone cell degeneration or preservation of cone cells in the retina of a mammal suffering from an ocular disease by intravitreally injecting the isolated stem cells of the invention into an eye of the mammal in a number sufficient to ameliorate the degeneration of cone cells in the retina.

From a clinical perspective, transplantation of the inventive cell shows that the actively degenerating adult retina can be repopulated with donor-derived neurons, including photoreceptors, that these new cells survive without exogenous immune suppression as well as exhibiting morphological evidence of integration with host circuitry.

The results achieved by the present invention show that the injected defined populations of inventive cells migrated to, incorporated into the retina, followed by proliferation and differentiation into retinal neural cells, i.e. neuronal repopulating areas of pathological cell loss within the retina. Where there is retinal degeneration of photoreceptors and retinal vascular layers, the present invention shows that intravitreally injected, transiently transfected cells in a defined population of the invention target specific cell types of the retina and participate in retinal regeneration (neuronal and, indirectly via FGF-2, the vasculature). The donor inventive cells stably incorporate into and promote angiogenesis in the injured retinal vasculature. The invention rescues cones and vasculature.

The cells and defined cell populations of the present invention secreting growth factors are employed as therapeutic agents. Generally, the transplantation methods herein involve transferring donor defined populations of inventive cells to desired tissue, either in vitro (e.g., as a graft prior to implantation or engrafting) or in vivo, to animal tissue directly. The cells can be transferred to the desired tissue by any method appropriate, which generally will vary according to the tissue type. For example, cells can be transferred to a graft by bathing the graft (or infusing it) with culture medium containing the cells. Alternatively, the cells can be seeded onto the desired site within the tissue to establish a population. Cells can be transferred to sites in vivo using devices such as catherters, trocars, cannulae, stents (which can be seeded with the cells), etc. For these applications, preferably the cell secretes a cytokine or growth hormone such as human growth factor, fibroblast growth factor, nerve growth factor, insulin-like growth factors, hemopoetic stem cell growth factors, members of the fibroblast growth factor family, members of the platelet-derived growth factor family, vascular and endothelial cell growth factors, members of the TGFb family (including bone morphogenic factor), or enzymes specific for congenital disorders (e.g., distrophin).

All references to U.S. patent literature are hereby incorporated by reference, as are, to the extent possible, the literature cited herein.

### CITED LITERATURE

1. H Klassen, D S Sakaguchi, M J Young: Stem cells and retinal repair. Prog Retin Eye Res 2004, 23:149-81.

2. L E Smith: Bone marrow-derived stem cells preserve cone vision in retinitis pigmentosa. J Clin Invest 2004, 114:755-7.

3. M J Young, J Ray, S J Whiteley, H Klassen, F H Gage: Neuronal differentiation and morphological integration of hippocampal progenitor cells transplanted to the retina of immature and mature dystrophic rats. Mol Cell Neurosci 2000, 16:197-205.

4. M Tomita, Y Adachi, H Yamada, K Takahashi, K Kiuchi, H Oyaizu, K Ikebukuro, H Kaneda, M Matsumura, S Ikehara: Bone marrow-derived stem cells can differentiate into retinal cells in injured rat retina. Stem Cells 2002, 20:279-83.

5. A Otani, K Kinder, K Ewalt, F J Otero, P Schimmel, M Friedlander: Bone marrow-derived stem cells target retinal astrocytes and can promote or inhibit retinal angiogenesis. Nat Med 2002, 8:1004-10.

6. P A Zuk, M Zhu, P Ashjian, D A De Ugarte, J I Huang, H Mizuno, Z C Alfonso, J K Fraser, P Benhaim, M H Hedrick: Human Adipose Tissue Is a Source of Multipotent Stem Cells. Mol. Biol. Cell 2002, 13:4279-4295.

7. L Conti, S M Pollard, T Gorba, E Reitano, M Toselli, G Biella, Y Sun, S Sanzone, Q L Ying, E Cattaneo, et al: Niche-independent symmetrical self-renewal of a mammalian tissue stem cell. PLoS Biol 2005, 3:e283.

8. S C Zhang, M Wernig, I D Duncan, O Brustle, J A Thomson: In vitro differentiation of transplantable neural precursors from human embryonic stem cells. Nat Biotechnol 2001, 19:1129-33.

9. A Otani, M I Dorrell, K Kinder, S K Moreno, S Nusinowitz, E Banin, J Heckenlively, M Friedlander: Rescue of retinal degeneration by intravitreally injected adult bone marrow-derived lineage-negative hematopoietic stem cells. J Clin Invest 2004, 114:765-74.

1. A method of treating retinal disease comprising the step of intravitreally injecting a stem cell population into the eye of a mammal, wherein said stem cell population comprises stem cells which comprise transient wound-healing cytokine transgenes.

2. The method of claim 1 wherein said stem cell population is an adipose-derived stem cell population alone or in combination with peripheral blood progenitor stem cells.

3. The method of claim 1 wherein said transient transgenes are selected from the group of pro-inflammatory and wound-healing cytokines consisting of IL-6, IL-12, LIF, IFN(s), EGF, VEGF, FGF-1, FGF-2.

4. The method of claim 3 wherein said transient transgenes are EGF and FGF-2.

5. An isolated adipose-derived stem cell comprising transient wound-healing trangenes.

6. The cell of claim 5 wherein said transient transgenes are selected from the group consisting of pro-inflammatory and wound-healing cytokines.

7. The cell of claim 6 wherein said pro-inflammatory and wound-healing cytokines include IL-6, IL-12, LIF, IFN(s), EGF, VEGF, FGF-1, FGF-2.

8. The cell of claim 7 wherein said transient transgene is EGF or FGF-2.

9. A substantially homogenous population of adipose-derived stem cells, said cells being transiently transfected with one or more wound-healing transgenes.

10. The population of claim 9 wherein said transgenes are selected from the group of pro-inflammatory and wound-healing cytokines, including IL-6, IL-12, LIF, IFN(s), EGF, VEGF, FGF-1, FGF-2.

11. The population claim 10 wherein said transient transgenes are EGF and FGF-2.

12. A method of treating retinal disease comprising the steps of:
a. treating the retina with a laser to induce retinal injury, and
b. intravitreally injecting a population of stem cells comprising transiently transfected stem cells into the eye of the mammal in a number sufficient for repopulation, intraretinal integration and differentiation into normal photoreceptor cells.

13. The method of claim 12 comprising the further step of exogenously providing through intravitreally injection a growth factor to said injected population of stem cells.

14. The method of claim 12 wherein said laser treatment comprises contacting said retina with a wavelength of 1079 nm to create injury which comprises coagulation of deeper layers of retinal tissue with formation of wells and stromal scaffold formation and contacting said retina with a wavelength of 540 nm initiating angiogenesis.
